(19) United States
(12) Patent Application Publication
(10) Pub. No.: US 2024/0100356 A1
(43) Pub. Date: Mar. 28, 2024

(54) CUSTOMIZED BRACHYTHERAPY NEEDLE TEMPLATE AND METHOD

(71) Applicant: Cowles Ventures, LLC, Spokane, WA (US)

(72) Inventors: Emily Lin, Spokane, WA (US); Byron Stuck, Spokane, WA (US); Kevin Kelley, Spokane, WA (US)

(73) Assignee: Cowles Ventures, LLC, Spokane, WA (US)

(21) Appl. No.: 18/532,025

(22) Filed: Dec. 7, 2023

Related U.S. Application Data
(63) Continuation of application No. PCT/US2022/032636, filed on Jun. 8, 2022.
(60) Provisional application No. 63/208,348, filed on Jun. 8, 2021.

(51) Int. Cl. A61N 5/10

(52) U.S. Cl. CPC A61N 5/1007; A61N 2005/1012

(57) ABSTRACT

A customized template and method create a template that is customized for a particular patient based on a treatment plan of the patient to perform a brachytherapy treatment.

### RELATED APPLICATIONS

This application claims the benefit under 35 USC 120 and is a continuation of Application Serial No. PCT/US2022/032636 filed Jun. 8, 2022 that in turn claims priority under and the benefit of 35 USC 119(e) of U.S. Provisional Application No. 63/208,348, filed Jun. 8, 2021 and which is incorporated herein by reference.

### FIELD

The disclosure is directed to a customized needle template for brachytherapy.

### BACKGROUND

Brachytherapy is a medical procedure in which one or more radioactive sources may be inserted into a body cavity or body tissue (collectively “target region”) of a patient in order to irradiate certain tissue with localized radiation from the inserted radioactive sources. The radiation from the radioactive sources may be used to treat diseases such as cancer and the like. In order to insert the one or more radioactive sources into the target region, a dosimetry determination must be done to determine a number of radioactive sources to be inserted to achieve the desired radiation dosage to the target region and the number of needles with containing the radioactive sources that are used to insert the one or more radioactive sources.

Once the number of radioactive sources and the number of needles are determined, a medical procedure may be performed to insert the one or more radioactive sources, such as radioactive seeds, into specific locations within the target region to deliver the desired radiation dose to the tissue. During the medical procedure, one or more fiducial needles are inserted and used to ensure that the radioactive sources are implanted into the specific locations and then the radioactive sources are dispensed from radioactive source needles to the specific locations. In some medical procedures, a template may be used that holds the one or more fiducial needles embedded in the target region fixed relative to a fixed frame of reference so that the radioactive source needles may be inserted through the template and guided by the template to the specific locations in the target region. As a result, the holes through which the template that the radioactive source needles and the one or more fiducial needles pass must be very precise. An example of a device with a template that may be used for brachytherapy is U.S. Pat. No. 8,764,619 that is incorporated herein by reference.

Most of the templates used in brachytherapy have a coordinate grid system such that each hole of the template may be identified by vertical and horizontal coordinates. The coordinate system is a way to implement the treatment plan and guide the insertion of the radioactive source needles and fiducial needles into their designated coordinates. The user carefully matches the radioactive source needles and fiducial needles to their designated coordinates and location. Misplacement of a radioactive source could result in the wrong radiation dose. It is desirable to simplify the process of inserting the radioactive source needles and fiducial needles into the correct location to maintain the precision of radiation delivery. A template that is customized according to a treatment plan will reduce the risk of inserting a radioactive source needle or fiducial needle in the wrong location. It is also necessary that the template, even if customized, maintains the precise alignment of the holes through which the fiducial needles and the radioactive source needles pass during the procedure.

### BRIEF DESCRIPTION OF THE DRAWINGS

FIGS. 1A and 1B show a brachytherapy system that may be used for breast brachytherapy;

FIGS. 2 and 3 illustrates a first embodiment of a customized template having a template and a template cover with a set number of holes for needle insertion;

FIG. 4 illustrates a method for using the customized template shown in FIGS. 2 and 3;

FIG. 5 illustrates a second embodiment of the custom template with a set number of holes for needle insertion; and

FIG. 6 illustrates a method for using a customized template shown in FIG. 5.

### DETAILED DESCRIPTION OF ONE OR MORE EMBODIMENTS

The disclosure is particularly applicable to a customized template for use in a breast tissue brachytherapy procedure using palladium radioactive seeds using the brachytherapy device disclosed in U.S. Pat. No. 8,764,619 and it is in this context that the disclosure will be described. It will be appreciated, however, that the device and method has greater utility because: 1) the customized template may be used for various different brachytherapy procedure; 2) the customized template may be used with different radioactive sources other than the palladium radioactive seeds; and 3) the customized template may be used with other brachytherapy devices. Before describing the customized template and method, a brachytherapy system in which the customized template may be used is described.

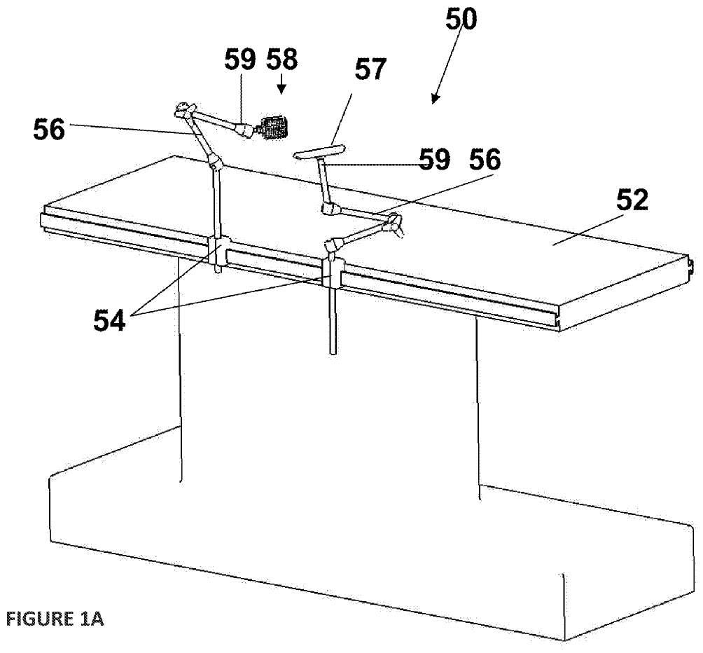

FIGS. 1A and 1B show a brachytherapy system 50 that may be used for breast brachytherapy (also known as adjuvant partial breast irradiation.) The mechanism 50 may include a table 52 on which a patient lies of their back to have the radioactive sources implanted into the surgery cavity in their breast tissue following breast conservation surgery. The mechanism may also include one or more well known table clamps 54 that are used to clamp one or more stereotactic armatures 56 to the table and fix the stereotactic armatures relative to the table. Each stereotactic armature 56 may have joints that allow the position of an operational end 59 of the armature to be adjusted and then fixed relative to the table in three dimensions to form a fixed frame of reference. The mechanism may further include a needle retraction stop 57 attached to one stereotactic armature 56 and a fiducial needle fixation assembly 58 attached to another stereotactic armature 56 to form the fixed frame of reference.

As shown in FIG. 1B, the fiducial needle fixation assembly 58 may further include an engagement device 60 that allows a fiducial needle fixation device (a template) 62 to be quickly and securely removably fixed to the stereotactic armature 56. Generally, the patient may lie on the table with the breast tissue against the fiducial needle fixation assembly 58 so that one or more fiducial needles may be inserted into the breast tissue and the surgery cavity using an electromagnetic imaging system, such as for example ultrasound so that the surgeon can then accurately place the one or more radioactive sources into the tumor bed/surgery cavity using an electromagnetic imaging system, such as for example ultrasound to visualize the tumor bed/surgery cavity due to the one or more fiducial needles as described below in more detail. In one implementation, the one or more radioactive sources may be ¹⁰³PD seeds and/or seed strands. The radioactive seeds may also be ¹²⁵I and or ¹³¹Cs.

The template 62 may be a solid/hollow rectangular shaped piece of material (although the template can be other shapes and sizes to accommodate differing tumor/patient sizes) that has the lockable fiducial needle locations 108 as well as the other needle holes that are not lockable as described above. The template 62 may be made of plastic, metal, etc. Once the one or more fiducial needles are locked into position and the fiducial needle fixation device 62 is locked into position relative to the mechanism 50, the needles with the radioactive sources can be inserted into the template holes at precisely known locations to insert and implant the radioactive sources into the breast tissue according to the coordinated locations of the prescription treatment plan.

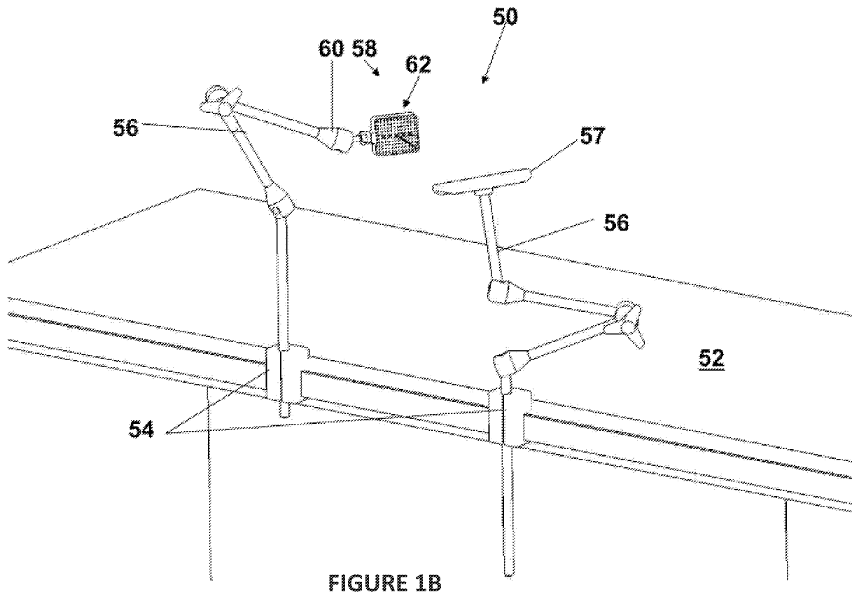

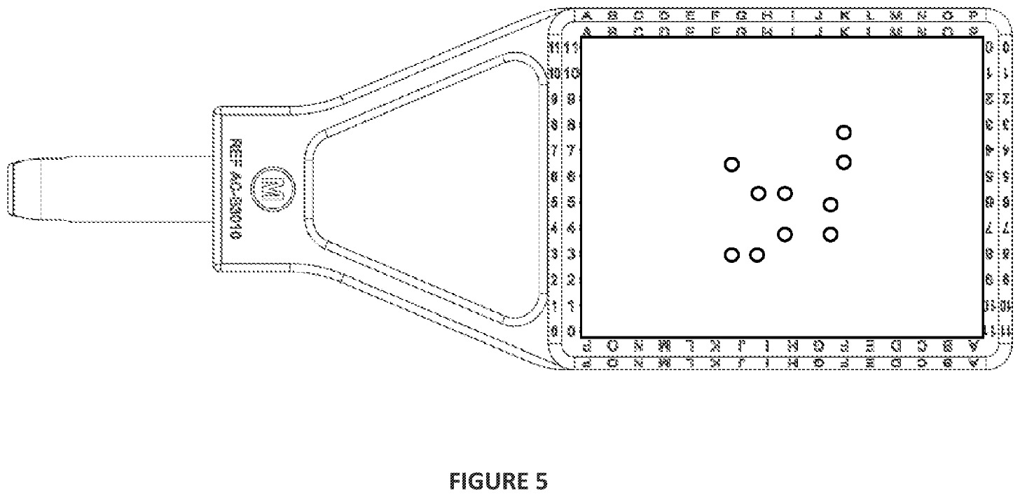

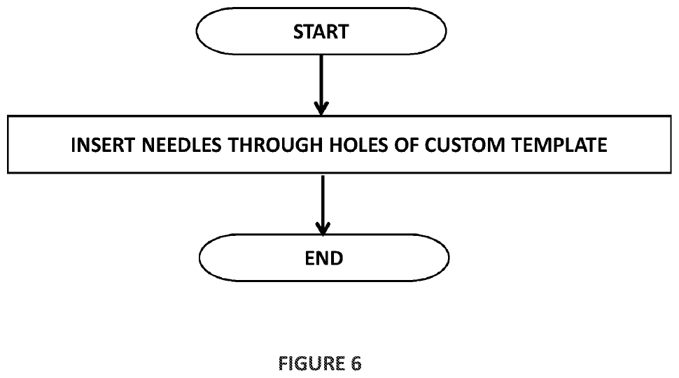

The system and method described provides a quality control mechanism to guide the insertion of the radioactive source needles and can be implemented using the various devices and mechanisms described below. Two embodiments of the template are disclosed with one embodiment shown in FIGS. 2-4 and the second embodiment shown in FIGS. 5-6. Note that although top plan views of the customizable template are shown in FIGS. 2 and 5, is a piece of material that has a depth (as shown in FIG. 1B) and the bottom side of the template may be contoured to fit against a particular patient with a particular breast size/shape.

As shown in FIG. 2, the template 62 may have an attachment arm 200 that fits into the fixation mechanism shown in FIGS. 1A and 1B such that the template 62 is fixated relative to the breast into which radioactive seeds are being placed. The template 62 also has a triangular shaped arm 202 that connects the arm 200 to a template matrix 204. The template matrix 204 may have a rectangular array of holes (labeled as A11, P0, for example) through which needles with radioactive seeds may be passed to accurately and precisely insert the radioactive seeds into the breast tissue of the patient since the template matrix 204 is fixated relative to the fixation mechanism and thus the surgical table and the patient. The template matrix 204 may also have one or more center lines holes (denoted by the plus signs in FIG. 2) through which fiducial needles may be placed into the breast tissue. The fiducial needles once inserted through the template matrix 204 may be locked in place so that fiducial needles are fixated relative to the template matrix 204. Once those fiducial needles are locked into place, the grid of holes for the radioactive seed needles may be threaded through the holes into precise positions in the breast tissue.

In most conventional brachytherapy treatments, the template is generic and treatment software/a radiation oncologist may determine the holes in the template through which the radioactive seed needles may be inserted. Using the customized template 62, the template 62 may be customized to the treatment plan so that it only contains holes through which needles will pass. In this way, the only template holes open for needles to pass through are based on the treatment plan. Thus, a treatment plan for the particular patient is done in a known manner that is done before each brachytherapy treatment. In the first embodiment, the template 62 may include a template cover 300 that has selected holes in it based on the treatment plan through which needles may pass. When the template cover 300 is attached to the template 62 (that may itself be un-customized and has not been customized to the treatment plan, the template cover blocks the holes of the template that are not part of the treatment plan. In this way, only the holes of the treatment plan are open for needles to pass through to insert the radioactive seeds based on the treatment plan. In this first embodiment, a general template may be customized using the template cover 300.

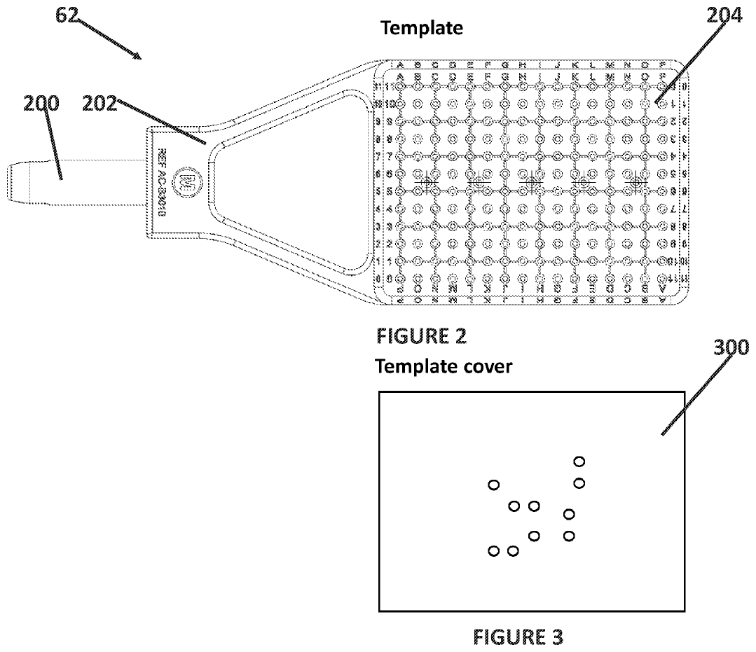

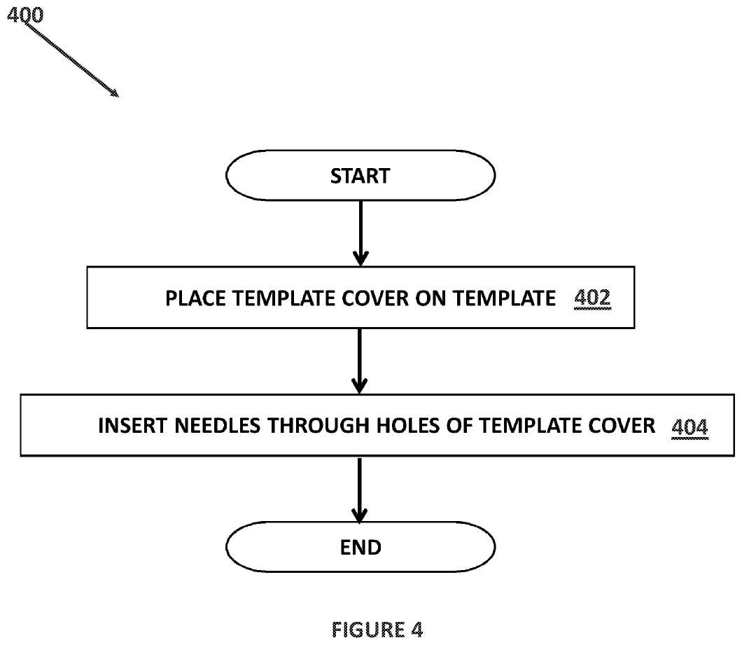

FIG. 4 illustrates a method 400 of customized brachytherapy treatment using the customized template in FIGS. 2-3. First, holes are made in the template cover per the treatment plan and the template cover may be placed on the template (402). The person performing the brachytherapy then inserts radioactive seed needles (a needle that holds one or more radioactive seeds that may be placed into the patient) through the holes in the template cover to place the radioactive seeds/sources per the treatment plan customized to the patient.

FIG. 5 shows a second embodiment of the customized template that has the same elements as the first embodiment. In this embodiment, however, the template only has holes that are customized for the treatment plan of the particular patient. FIG. 6 shows the method in which the radioactive seed needles are inserted through the holes in the customized template to perform the brachytherapy treatment.

In each of the above embodiments or other embodiments, the customized template may have light and color indicators for each hole, which are programmed to turn on or change based on the treatment plan. For example, the holes of the template that are part of the treatment plan light up to indicate the insertion site. Furthermore, the customized template may contain sensors that can recognize when the correct needle is through the correct hole. The template can output different light and/or sound indicators depending on whether the right or wrong insertion site is used. A laser or virtual display of the needle insertion sites is displayed on the body. Alternatively, augmented reality can be used to the overlay the treatment plan over the breast. Additional visual guidance on the treatment plan, needle insertion angle, and point of entry may be provided via augmented reality.

The foregoing description, for purpose of explanation, has been with reference to specific embodiments. However, the illustrative discussions above are not intended to be exhaustive or to limit the disclosure to the precise forms disclosed. Many modifications and variations are possible in view of the above teachings. The embodiments were chosen and described in order to best explain the principles of the disclosure and its practical applications, to thereby enable others skilled in the art to best utilize the disclosure and various embodiments with various modifications as are suited to the particular use contemplated.

The system and method disclosed herein may be implemented via one or more components, systems, servers, appliances, other subcomponents, or distributed between such elements. When implemented as a system, such systems may include and/or involve, inter alia, components such as software modules, general-purpose CPU, RAM, etc. found in general-purpose computers. In implementations where the innovations reside on a server, such a server may include or involve components such as CPU, RAM, etc., such as those found in general-purpose computers.

Additionally, the system and method herein may be achieved via implementations with disparate or entirely different software, hardware and/or firmware components, beyond that set forth above. With regard to such other components (e.g., software, processing components, etc.) and/or computer-readable media associated with or embodying the present inventions, for example, aspects of the innovations herein may be implemented consistent with numerous general purpose or special purpose computing systems or configurations. Various exemplary computing systems, environments, and/or configurations that may be suitable for use with the innovations herein may include, but are not limited to: software or other components within or embodied on personal computers, servers or server computing devices such as routing/connectivity components, hand-held or laptop devices, multiprocessor systems, microprocessor-based systems, set top boxes, consumer electronic devices, network PCs, other existing computer platforms, distributed computing environments that include one or more of the above systems or devices, etc.

In some instances, aspects of the system and method may be achieved via or performed by logic and/or logic instructions including program modules, executed in association with such components or circuitry, for example. In general, program modules may include routines, programs, objects, components, data structures, etc. that perform particular tasks or implement particular instructions herein. The inventions may also be practiced in the context of distributed software, computer, or circuit settings where circuitry is connected via communication buses, circuitry or links. In distributed settings, control/instructions may occur from both local and remote computer storage media including memory storage devices.

The software, circuitry and components herein may also include and/or utilize one or more type of computer readable media. Computer readable media can be any available media that is resident on, associable with, or can be accessed by such circuits and/or computing components. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical storage, magnetic tape, magnetic disk storage or other magnetic storage devices, or any other medium which can be used to store the desired information and can accessed by computing component. Communication media may comprise computer readable instructions, data structures, program modules and/or other components. Further, communication media may include wired media such as a wired network or direct-wired connection, however no media of any such type herein includes transitory media. Combinations of the any of the above are also included within the scope of computer readable media.

In the present description, the terms component, module, device, etc. may refer to any type of logical or functional software elements, circuits, blocks and/or processes that may be implemented in a variety of ways. For example, the functions of various circuits and/or blocks can be combined with one another into any other number of modules. Each module may even be implemented as a software program stored on a tangible memory (e.g., random access memory, read only memory, CD-ROM memory, hard disk drive, etc.) to be read by a central processing unit to implement the functions of the innovations herein. Or, the modules can comprise programming instructions transmitted to a general-purpose computer or to processing/graphics hardware via a transmission carrier wave. Also, the modules can be implemented as hardware logic circuitry implementing the functions encompassed by the innovations herein. Finally, the modules can be implemented using special purpose instructions (SIMD instructions), field programmable logic arrays or any mix thereof which provides the desired level performance and cost.

As disclosed herein, features consistent with the disclosure may be implemented via computer-hardware, software, and/or firmware. For example, the systems and methods disclosed herein may be embodied in various forms including, for example, a data processor, such as a computer that also includes a database, digital electronic circuitry, firmware, software, or in combinations of them. Further, while some of the disclosed implementations describe specific hardware components, systems and methods consistent with the innovations herein may be implemented with any combination of hardware, software and/or firmware. Moreover, the above-noted features and other aspects and principles of the innovations herein may be implemented in various environments. Such environments and related applications may be specially constructed for performing the various routines, processes and/or operations according to the invention or they may include a general-purpose computer or computing platform selectively activated or reconfigured by code to provide the necessary functionality. The processes disclosed herein are not inherently related to any particular computer, network, architecture, environment, or other apparatus, and may be implemented by a suitable combination of hardware, software, and/or firmware. For example, various general-purpose machines may be used with programs written in accordance with teachings of the invention, or it may be more convenient to construct a specialized apparatus or system to perform the required methods and techniques.

Aspects of the method and system described herein, such as the logic, may also be implemented as functionality programmed into any of a variety of circuitry, including programmable logic devices (“PLDs”), such as field programmable gate arrays (“FPGAs”), programmable array logic (“PAL”) devices, electrically programmable logic and memory devices and standard cell-based devices, as well as application specific integrated circuits. Some other possibilities for implementing aspects include: memory devices, microcontrollers with memory (such as EEPROM), embedded microprocessors, firmware, software, etc. Furthermore, aspects may be embodied in microprocessors having software-based circuit emulation, discrete logic (sequential and combinatorial), custom devices, fuzzy (neural) logic, quantum devices, and hybrids of any of the above device types. The underlying device technologies may be provided in a variety of component types, e.g., metal-oxide semiconductor field-effect transistor (“MOSFET”) technologies like complementary metal-oxide semiconductor (“CMOS”), bipolar technologies like emitter-coupled logic (“ECL”), polymer technologies (e.g., silicon-conjugated polymer and metal-conjugated polymer-metal structures), mixed analog and digital, and so on.

It should also be noted that the various logic and/or functions disclosed herein may be enabled using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied include, but are not limited to, non-volatile storage media in various forms (e.g., optical, magnetic or semiconductor storage media) though again does not include transitory media. Unless the context clearly requires otherwise, throughout the description, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

Although certain presently preferred implementations of the invention have been specifically described herein, it will be apparent to those skilled in the art to which the invention pertains that variations and modifications of the various implementations shown and described herein may be made without departing from the spirit and scope of the invention. Accordingly, it is intended that the invention be limited only to the extent required by the applicable rules of law.

While the foregoing has been with reference to a particular embodiment of the disclosure, it will be appreciated by those skilled in the art that changes in this embodiment may be made without departing from the principles and spirit of the disclosure, the scope of which is defined by the appended claims.

What is claimed is:

1. A device, comprising:
a rectangular brachytherapy template being made of a piece of material having a rectangular array of holes through which a radioactive source needle is capable of passing;
a predetermined number of holes being open according to a treatment plan for a particular patient and each hole that is not one of the predetermined number of holes being closed; and
wherein the brachytherapy template is customized to the treatment plan for the particular patient.

2. The device of claim 1 further comprising a cover that covers each hole that is not one of the predetermined number of holes and leaves open the predetermined number of holes to create the customized template.

3. The device of claim 1 further comprising a set of holes along a centerline of the customized template through which fiducial needles are inserted, each hole in the centerline set of holes capable of being locking the inserted fiducial needle to fixate the customized template relative to the patient.

4. The device of claim 1 further comprising one or more indicators that indicate a correct hole in the customized template to insert a source needle.

5. The device of claim 4, wherein the one or more indicators are lights.

6. The device of claim 4, wherein the one or more indicators are colors.

7. A method, comprising:
performing treatment planning for a brachytherapy treatment for a particular patient, the treatment planning including determining a placement of each radioactive source in the patient;
creating a customized template based on the treatment plan, the customized template having a predetermined number of holes being open according to the treatment plan for the particular patient and each hole that is not one of the predetermined number of holes being closed; and using the customized template to perform a brachytherapy treatment for the particular patient.

8. The method of claim 7, wherein creating the customized template further comprises creating a cover that closes each hole that is not one of the predetermined number of holes and leaves open the predetermined number of holes to create the customized template.

9. The method of claim 8 further comprising placing the cover over the template.

10. The method of claim 7 further comprising fixating the customized template to a surgical table on which the particular patient is lying.

11. The method of claim 10 further comprising performing breast brachytherapy to insert radioactive sources into breast tissue on a patient.

12. The method of claim 11, wherein fixating the customized template inserting one or more fiducial needles through a set of holes along a centerline of the customized template into the breast tissue and locking each fiducial needle into a hole in the set of holes along the centerline of the customized template to fixate the customized template to the surgical table.

13. The method of claim 12 further comprising inserting a source needle having at least one radioactive source into one of the open holes in the customized template and depositing the at least one radioactive source in the breast tissue.

14. The method of claim 13, wherein the at least one radioactive source is palladium.
